(19) United States
(12) Patent Application Publication
(10) Pub. No.: US 2002/0156106 A1
(43) Pub. Date: Oct. 24, 2002

(54) Use of insulin sensitisers for treating renal diseases

(75) Inventor: Robin Edwin Buckingham, Welwyn Garden City (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/127,991

(22) Filed: Apr. 22, 2002

Related U.S. Application Data
(63) Continuation of application No. 09/863,534, filed on May 23, 2001, now abandoned.
Application No. 09/863,534 is a continuation of application No. 09/329,211, filed on Jun. 10, 1999, now abandoned.
Application No. 09/329,211 is a continuation of application No. 08/693,190, filed on Oct. 11, 1996, now abandoned.
Application No. 08/693,190 is a 371 of international application No. PCT/EP95/00441, filed on Feb. 7, 1995.

(30) Foreign Application Priority Data
Feb. 10, 1994 (GB) 9402624.2
May 21, 1994 (GB) 9410214.2
Dec. 22, 1994 (GB) 9426019.7

(51) Int. Cl. A61K 31/4439; A61K 31/426

(52) U.S. Cl. 514/342; 514/369

(57) ABSTRACT

A method of treatment and/or prophylaxis of renal diseases including diabetic nephropathy, glomerulonephritis, glomerular sclerosis, nephrotic syndrome, hypertensive nephrosclerosis and end stage renal disease, and micoralbuminuria which comprises the administration of an effective, non-toxic amount of an insulin sensitizer to a human or non-human mammal in need thereof.

This invention relates to a novel method for the treatment of renal diseases.

European Patent Applications, Publication Numbers: 0306228, 0008203, 0139421, 0032128, 0428312, 0489663, 0155845, 0257781, 0208420,0177353, 0319189, 0332331, 0332332,0528734, 0508740; International Patent Application, Publication Numbers 92/18501, 93/02079, 93/22445 and U.S. Pat. No. 5,104,888, disclose certain thiazolidinedione derivatives which are disclosed as having hypoglycaemic and hypolipidaemic activity. The thiazolidinedione derivatives disclosed in these patent applications are examples of a class of hypoglycaemic agent generally referred to as ‘insulin sensitisers’ and hence these compounds are referred to herein as ‘thiazolidinedione insulin sensitisers’.

Another series of compounds generally recognised as having insulin sensitiser activity are those typified by the compounds disclosed in International Patent Applications, Publication Numbers WO93/21166 and WO94/01420. These compounds are herein referred to as ‘acyclic insulin sensitisers’. Other examples of acyclic insulin sensitisers are those disclosed in U.S. Pat. No. 5,232,945 and International Patent Applications, Publication Numbers WO92/03425 and WO91/19702.

Examples of other insulin sensirisers are those disclosed in European Patent Application, Publication Number 0533933, Japanese Patent Application Publication Number 05271204 and U.S. Pat. No. 5,264,451.

We have now discovered that compounds having insulin sensitiser activity can prevent hydronephrosis and proteinuria, such as albuminuria, and that they are therefore of potential use in the treatment and/or prophylaxis of renal disease, especially renal disease associated with the development of Type II diabetes including diabetic nephropathy, glomerulonephritis, glomerular sclerosis, nephrotic syndrome, hypertensive nephrosclerosis and end stage renal disease. The prophylactic action of an insulin sensitiser upon nephropathy is also indicative that an insulin sensitising agent can be expected to prevent, reverse, stabilise or retard the progression of microalbuminuria to albuminuria. This is because microalbuminuria is considered to be a predictor of future nephropathy, especially in patients with clinical evidence of pre-diabetic insulin resistance syndrome, alternatively referred to as Syndrome X.

Accordingly, the present invention provides a method for the treatment and/or prophylaxis of renal diseases including diabetic nephropathy, glomerulonephritis, glomerular sclerosis, nephrotic syndrome, hypertensive nephrosclerosis and end stage renal disease, and microalbuminuria which method comprises the administration of an effective, non-toxic amount of an insulin sensitiser to a human or non-human mammal in need thereof.

Particular insulin sensitisers include thiazolidinedione insulin sensitisers.

Particular insulin sensitisers include acyclic insulin sensitisers.

One favoured group of insulin sensitisers are the thiazolidinedione insulin sensitisers disclosed in EP 0306228 and WO94/05659. Thus in a favoured aspect the present invention provides a method for the treatment and/or prophylaxis of renal diseases including diabetic nephropathy, glomerulonephritis, glomerular sclerosis, nephrotic syndrome, hypertensive nephrosclerosis and end stage renal disease, and microalbuminuria which method comprises the administration of an effective non-toxic amount of a compound of formula (I):

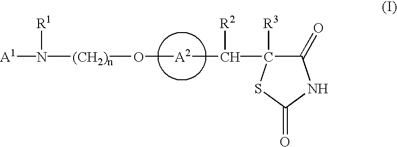

or a tautomeric form thereof and/or a pharmaceutically acceptable salt thereof, and/or a pharmaceutically acceptable solvate thereof, wherein:

A¹ represents a substituted or unsubstituted aromatic heterocyclyl group;

R¹ represents a hydrogen atom, an alkyl group, an acyl group, an aralkyl group, wherein the aryl moiety may be substituted or unsubstituted, or a substituted or unsubstituted aryl group;

R² and R³ each represent hydrogen, or R² and R³ together represent a bond;

A² represents a benzene ring having in total up to five substituents; and

n represents an integer in the range of from 2 to 6; to a human or non-human mammal in need thereof.

Suitable aromatic heterocyclyl groups of the compounds of formula (I) include substituted or unsubstituted, single or fused ring aromatic heterocyclyl groups comprising up to 4 hetero atoms in each ring selected from oxygen, sulphur or nitrogen.

Favoured aromatic heterocyclyl groups of the compounds of formula (I) include substituted or unsubstituted single ring aromatic heterocyclyl groups having 5 to 7 ring atoms, preferably 5 or 6 ring atoms.

In particular, the aromatic heterocyclyl groups of the compounds of formula (I) comprise 1, 2 or 3 heteroatoms, especially 1 or 2, selected from oxygen, sulphur or nitrogen.

Suitable values for A¹ when it represents a 5-membered aromatic heterocyclyl group include thiazolyl and oxazolyl, especially oxazolyl.

Suitable values for A¹ when it represents a 6-membered aromatic heterocyclyl group include pyridyl or pyrimidinyl, especially pyridyl.

Preferably, A¹ represents a moiety of formula (a), (b) or (c):

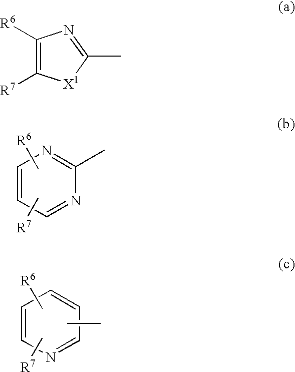

wherein:

R⁶ and R⁷ each independently represents a hydrogen or halogen atom, an alkyl or alkoxy group or a substituted or unsubstituted aryl group or when R⁶ and R⁷ are each attached to adjacent carbon atoms, then R⁶ and R⁷ together with the carbon atoms to which they are attached form a benzene ring wherein each carbon atom represented by R⁶ and R⁷ together is substituted or unsubstituted; and in the moiety of formula (a) X¹ represents oxygen or sulphur.

Aptly, A¹ represents a moiety of the abovedefined formula (a).

Aptly, A¹ represents a moiety of the abovedefined formula (b).

Aptly, A¹ represents a moiety of the abovedefined formula (c).

A particular form of moiety (c) is a moiety (c′):

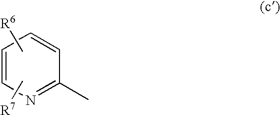

wherein R⁶ and R⁷ are as defined in relation to formula (c).

In one favoured aspect R⁶ and R⁷ together represent a moiety of formula (d):

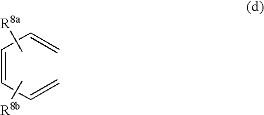

wherein R^{8a} and R^{8b} each independently represent hydrogen, halogen, substituted or unsubstituted alkyl or alkoxy.

Suitably, R^{8a} and R^{8b} each independently represent hydrogen, halogen, alkyl or alkoxy. Favourably, R^{8a} represents hydrogen. Favourably, R^{8b} represents hydrogen. Preferably, R^{8a} and R^{8b} both repersent hydrogen.

In a further favoured aspect R⁶ and R⁷ each independently represent hydrogen, alkyl or a substituted or unsubstituted phenyl group and more favourably, R⁶ and R⁷ each independently represent hydrogen, alkyl or phenyl.

Preferably, for the moiety of formula (a), R⁶ and R⁷ together represent the moiety of formula (d).

Preferably, for the moieties of formula (b), (c) or (c′), R⁶ and R⁷ both represent hydrogen. It will be appreciated that the five substituents of A² include three optional substituents. Suitable optional substituents for the moiety A² include halogen, substituted or unsubstituted alkyl or alkoxy.

Further suitable. favoured and preferred values for variables A², R¹, R², R³ and n are as defined in EP 0306228 and WO94/05659.

A preferred compound of formula (I) is 5-[4-[2-(N-methyl-N-(2-pyridyl) amino)ethoxy]benzyl]thiazolidine-2,4-dione or a tautomeric form thereof and/or a pharmaceutically acceptable salt thereof, especially a maleic acid salt thereof, and/or a pharmaceutically acceptable solvate thereof.

As indicated above, a compound of formula (I) may exist in one of several tautomeric forms, all of which are encompassed in the method of the present invention. It will be appreciated that the present invention encompasses the administration of all of the isomeric forms of the compounds of formula (I) and the pharmaceutically acceptable salts thereof, including any stereoisomeric forms thereof, whether as individual isomers or as mixtures of isomers.

Suitable substituents for any heterocyclyl group of the compounds of formula (I) include up to 4 substituents selected from the group consisting of: alkyl, alkoxy, aryl and halogen or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

The suitable, favoured and preferred thiazolidinedione insulin sensitisers disclosed in European Patent Applications, Publication Numbers: 0306228, 0008203, 0139421, 0032128, 0428312, 0489663, 0155845, 0257781, 0208420, 0177353, 0319189, 0332331,0332332, 0528734, 0508740; International Patent Application, Publication Numbers 92/18501, 93/02079, 93/22445 and U.S. Pat. No. 5,104,888 are those compounds defined as suitable, favoured and preferred in the respective patent publications.

The suitable, favoured and preferred acyclic insulin sensitisers disclosed in International Patent Applications, Publication Numbers WO91/19702, WO92/03425, WO93/21166 and WO94/01420 and U.S. Pat. No. 5,232,945 are those compounds defined as suitable, favoured and preferred in the respective patent publications.

Other suitable, favoured and preferred insulin sensitisers are the suitable, favoured and preferred compounds disclosed in European Patent Application, Publication Number 0533933, International Patent Application, Publication Number WO 93/02079 , Japanese Patent Application Publication Number 05271204 and U.S. Pat. No. 5,264,451.

Also specifically included in the method of the invention are the specific examples disclosed in the above mentioned patent applications.

When used herein the term ‘insulin sensitiser’ relates to compounds which increase the biological response to insulin. In addition, based upon the observed effects in appropriate test animals such as Zucker fatty (fa/fa) rats, insulin sensitisers are indicated to lower elevated fasting plasma insulin concentrations and improve glycaemic control.

When used herein the term ‘aryl’ includes phenyl and naphthyl optionally substituted with up to five, preferably up to three, groups selected from halogen, alkyl, phenyl, alkoxy, haloalkyl, hydroxy, amino, nitro, carboxy, alkoxycarbonyl, alkoxycarbonylalkyl, alkylcarbonyloxy, or alkylcarbonyl groups.

When used herein the tern ‘halogen’ refers to fluorine, chlorine, bromine and iodine; preferably chlorine.

When used herein the terms ‘alkyl’ and ‘alkoxy’ relate to groups having straight or branched carbon chains,containing up to 12 carbon atoms.

When used herein the term ‘acyl’ includes alkylcarbonyl groups.

Suitable alkyl groups are C₁₋₁₂ alkyl groups, especially C₁₋₆ alkyl groups e.g. methyl, ethyl, n-propyl, iso-propyl, n-butyl, isobutyl or tert-butyl groups.

Suitable substituents for any alkyl group include those indicated above in relation to the term “aryl”.

Suitable pharmaceutically acceptable salts include salts of carboxy groups and acid addition salts.

Suitable pharmaceutically acceptable salts of carboxy groups include metal salts, such as for example aluminium, alkali metal salts such as lithium, sodium or potassium, alkaline earth metal salts such as calcium or magnesium and ammonium or substituted ammonium salts, for example those with lower alkylamines such as trierhylamine, hydroxy alkylamines such as 2-hydroxyethylarnine, bis-(2-hydroxyethyl)-amine or tri-(2-hydroxyethyl)-amine, cycloalkylamines such as bicyclohexylamine, or with procaine, dibenzylpiperidine, N-benzyl-b-phenethylamine, dehydroabietylamine, N,N′-bisdehydroabietylamine, glucamine, N-methylglucamine or bases of the pyridine type such as pyridine, collidine, quinine or quinoline.

Suitable acid addition salts include pharmaceutically acceptable inorganic salts such as the sulphate, nitrate, phosphate, borate, hydrochloride and hydrobromide and pharmaceutically acceptable organic acid addition salts such as acetate, tartrate, maleate, citrate, succinate, benzoate, ascorbate, methane-sulphonate, a-keto glurarate and a-glycerophosphate, especially the maleate salt.

Suitable pharmaceutically acceptable solvates include hydrates.

The active compounds disclosed in the above mentioned patent publications, and referred to herein as insulin sensitisers, including the specific examples disclosed therein, are conveniently prepared according to the methods disclosed in the said patent publications: Thus a compound of formula (I), or the tautomenc form thereof, and/or a pharmaceutically acceptable salt thereof, and/or a pharmaceutically acceptable solvate thereof, may be prepared using the processes described in EP 0306228 and WO94/05659.

The salts and/or solvates of the compounds of formula (I) may be prepared and isolated according to conventional procedures for example sodium salts may be prepared by using sodium methoxide in methanol.

The present invention also provides an insulin sensitiser,such as a compound of formula (I), or a tautomeric form thereof and/or a pharmaceutically acceptable salt thereof and/or a pharmaceutically acceptable solvate thereof, for use in the treatment of and/or prophylaxis of renal diseases including diabetic nephropathy, glomerulonephritis, glomerular sclerosis, nephrotic syndrome, hypertensive nephrosclerosis and end stave renal disease, and microalbuminuria.

The present invention also provides an insulin sensitiser,such as a compound of formula (I), or a tauromeric form thereof and/or a pharmaceutically acceptable salt thereof and/or a pharmaceutically acceptable solvate thereof, for use in the manufacture of a medicament for the treatment and/or prophylaxis of renal diseases including diabetic nephropathy, glomerulonephritis, glomerular sclerosis, nephrotic syndrome, hypertensive nephrosclerosis and end stage renal disease, and microalbuminuria.

In the above mentioned treatment and or prophylaxis the insulin sensitiser such as a compound of formula (I), or a tautomeric form thereof and/or a pharmaceutically acceptable salt thereof and/or a pharmaceutically acceptable solvate thereof, may be administered per se or, preferably, as a pharmaceutical composition also comprising a pharmaceutically acceptable carrier.

Accordingly, the present invention also provides a pharmaceutical composition for the treatment and/or prophylaxis of renal diseases including diabetic nephropathy, glomerulonephritis, glomerular sclerosis, nephrotic syndrome, hypertensive nephrosclerosis and end stage renal disease, and microalbuminuria which composition comprises an insulin sensitiser, such as a compound of the formula (I), or a tautomeric form thereof, or a pharmaceutically acceptable salt thereof, or a pharmaceutically acceptable solvate thereof, and a pharmaceutically acceptable carrier therefor.

As used herein the term ‘pharmaceutically acceptable’ embraces compounds, compositions and ingredients for both human and veterinary use: for example the term ‘pharmaceutically acceptable salt’ embraces a veterinarily acceptable salt.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Particularly suitable compositions for oral administration are unit dosage forms such as tablets and capsules. Other fixed unit dosage forms, such as powders presented in sachets, may also be used.

In accordance with conventional pharmaceutical practice the carrier may comprise a diluent, filler, disintegrant, wetting agent, lubricant, colourant, flavourant or other conventional adjuvant.

Typical carriers include, for example, microcrystalline cellulose, starch, sodium starch glycollate, polyvinylpyrrolidone, polyvinylpolypyrrolidone, magnesium stearate, sodium lauryl sulphate or sucrose.

Most suitably the composition will be formulated in unit dose form. Such unit dose will normally contain an amount of the active ingredient in the range of from 0.1 to 1000 mg, more usually 0.1 to 500 mg, and more especially 0.1 to 250 mg.

Conveniently, the active ingredient may be administered as a pharmaceutical composition hereinbefore defined, and this forms a particular aspect of the present invention.

In the above mentioned treatments an insulin sensitiser, such as a compound of the formula (I), or a tautomeric form thereof and/or a pharmaceutically acceptable salt thereof and/or a pharmaceutically acceptable solvate thereof, may be taken in doses, such as those described above, one to six times a day in a manner such that the total daily dose for a 70 kg adult will generally be in the range of from 0.1 to 6000 mg, and more usually about 1 to 1500 mg, generally about 0.5 to 10 mg. That is in the range of from 1.429×10⁻³ to 85.714 mg/kg/day, more usually about 1.429×10⁻² to 21.429 mg/kg/day, generally about 7.143×10⁻³ to 0.1429 mg/kg/day.

The following Examples illustrate the invention but do not limit it in any way.

### EXAMPLE 1

### Studies Into the Effects of Tile Test Compound Upon Renal Pathology

Obese Zucker rats are known to develop chronic nephropathy in addition to hyperlipidaemia, hyperinsulinaemia and peripheral insulin resistance (Kasiske et al 1985).

5-[4-[2-(N-methyl-N-(2-pyridyl)amino)ethoxy]benzyl]thiazolidine-2,4-dione (herein after referred to as ‘test compound’) was administered to 2-3 month old obese Zucker fa/fa rats by dietary administration to provide a daily dose over a period of 3 months ranging from 2.0-7.0 μmote/kg body weight. A group of age-matched obese Zucker fa/fa rats were given the same diet without the addition of the test compound, as was a group of lean Fa/? rats. At the end of the study, all of the control Zucker fa/fa rats had chronic nephropathy which involved dilated tubules, atrophied or hyperp lastic tubular epithelial cells, thickened tubular basement membranes, segmented glomeruli or global glomerulosclerosis.

In the corresponding lean animals, 3/15 animals, a minimal degree of chronic nephropathy was seen.

Treatment with test compound resulted in a reduction in the incidence and degree of chronic nephropathy compared to the control group of Zucker fa/fa rats.

In the kidneys of the control Zucker fa/fa rats, mild-moderate hydronephrosis was seen in 4/9 animals. Characteristically this involved dilation of the kidney pelvis. No hydronephrosis was seen in the rats treated with test compound.

TABLE OF SIGNIFICANT HISTOPATHOLOGICAL
FINDINGS IN THE KIDNEY OF FATTY ZUCKER RATS

| Group/Treatment | | |
|---|---|---|
| Animal Number | Hydronephrosis | Chronic Nephropathy |
| Group 1 | | |
| Test Compound in diet | | |
| 2 | 0 | ± |
| 7 | 0 | ± |
| 9 | 0 | 0 |
| 15 | 0 | ++ |
| 16 | 0 | ± |
| 17 | 0 | + |
| 21 | 0 | ± |
| 22 | 0 | ± |
| 24 | 0 | ± |
| 27 | 0 | ± |
| 28 | 0 | ± |
| Group 2 | | |
| Diet only | | |
| 3 | ++ | + |
| 5 | +++ | + |
| 13 | 0 | + |
| 14 | ++ | ++ |
| 18 | 0 | + |
| 23 | +++ | ++ |
| 26 | 0 | + |
| 29 | 0 | + |
| 30 | 0 | ++ |

Severity Key
0 None seen
±Minimal
+Mild
++Moderate
+++Marked

### EXAMPLE 2

### Studies Into the Effect of Test CornpoundsUpon Systolic Blood Pressure, Urinary Total Protein and Urinary N-Acetyl B-D-Glucosaminidase (NAG) Activity Measurements.

A second study was performed in Zucker rats over a period of 9 months to investigate the longitudinal effects of the drug on systolic blood pressure and various indices of renal function, two of which are exemplified here. In one arm of the study, the drug was given in the diet (50 μmole/kg of diet) from aged 6-7weeks in Zucker fatty (fa/fa) rats, whilst in a second arm of the study, drug treatment as above was delayed until proteinuria had become established after 4 months, indicative of structural damage already present in the kidneys. A third group of Zucker fatty rats and a further group of lean rats were given diet alone throughout the period of study.

After dosing with the rest compound, at monthly intervals measurements were made of systolic blood pressure, urinary total protein and urinary N-acetyl β-D-glucosaminidase (NAG) activity.

Measurement of Systolic Blood Pressure

Rats were enclosed in custom-built restrainers and placed on a shelf in a warming cabinet, whose temperature was controlled at approximately 30° C. An inflatable cuff with integral pulse sensor was attached to the tail of each rat. After warming for 20-30 min the tail cuff was inflated and deflated automatically and a measurement of systolic blood pressure made using an IITC Non-Invasive Blood Pressure Monitor. This cycle was repeated several times for each rat until stable values of blood pressure were obtained.

Measurement of Urinary Parameters

Twenty-four hour urine collections, made in metabolism cages, were aliquoted and frozen at −70° C. until required for assay.

(i) Urinary Protein

Urinary protein concentration was measured using the Bio-Rad protein assay as modified for use in a 96-well microtitre plate. The assay is a dye-binding assay based on the differential colour change of a dye in response to various concentrations of protein (Bradford, Anal. Biochem., 72, 248, 1976). After a period of incubation with Dye Reagrent, diluted urine samples are read at an optical density of 595 nm using a Molecular Devices multiplate reader.

(ii) Urinary NAG Activity

NAG activity was assayed using a reagent kit on a Hitachi 717 analyser (both suppied by Boehringer Mannheim UK, Lewes). Enzyme activity was measured by monitoring the rate of chlorophenol (570 nm) released from the substrate chlorophenol red-β-D-glucosaminide.

Results and Statistics

In the tables below, results are given as mean values for the group±the standard error of the mean. Results have been analysed by one way ANOVA and significant differences from the Zucker fatty rat control group have been indicated by an asterisk.

Conclusion

The results of Example 2 demonstrate that treatment of Zucker fatty (fa/fa) rats from the age of 6-7 weeks, for a period of 9 months, with BRL 49653 given via the diet, prevented the development of hypertension and markedly reduced both the elevation of urinary NAG activity and the rate of development of proteinuria. When drug treatment was commenced after proteinuria had become established, both systolic blood pressure and urinary NAG activity were prevented from rising further and again there was a marked reduction of the rate of increase in the urinary protein concentration.

### Effects of Starting Treatment with Test Compound Prior to the Development of Renal Complications

SYSTOLIC BLOOD PRESSURE (mm Hg)

| Treatment | Zucker fatty rats | Zucker fatty rats | Lean rats |
|---|---|---|---|
| Duration | Test Compound | Control | Control |
| (months) | (50 μmol/kg of diet) | (powdered chow) | (powdered chow) |
| 0 | 102 ± 3 | 106 ± 3 | 123 ± 3* |
| 1 | 110 ± 3* | 120 ± 4 | 128 ± 3 |
| 2 | 122 ± 4* | 137 ± 4 | 132 ± 4 |
| 3 | 122 ± 4* | 142 ± 6 | 134 ± 3 |
| 4 | 126 ± 6* | 146 ± 4 | 132 ± 4* |
| 5 | 131 ± 4* | 157 ± 6 | 138 ± 4* |
| 6 | 128 ± 3* | 150 ± 6 | 133 ± 2* |
| 7 | 125 ± 6* | 157 ± 6 | 128 ± 6* |
| 8 | 133 ± 5* | 155 ± 4 | 130 ± 3* |
| 9 | 143 ± 4* | 164 ± 4 | 136 ± 3* |

### Effects of Starting Treatment with Test Compound Prior to the Development of Renal Complications

URINARY PROTEIN CONCENTRATION (μg/hour)

| Treatment | Zucker fatty rats | Zucker fatty rats | Lean rats |
|---|---|---|---|
| Duration | Test Compound | Control | Control |
| (months) | (50 μmol/kg of diet) | (powdered chow) | (powdered chow) |
| 0 | 226 ± 15* | 287 ± 17 | 198 ± 19* |
| 1 | 373 ± 22 | 355 ± 18 | 321 ± 23* |
| 2 | 402 ± 22* | 509 ± 28 | 396 ± 20* |
| 3 | 221 ± 32* | 874 ± 102 | 393 ± 25* |
| 4 | 376 ± 54* | 3965 ± 731 | 613 ± 52* |
| 5 | 1573 ± 105* | 5823 ± 809 | 1395 ± 88* |
| 6 | 1585 ± 147* | 8946 ± 1171 | 1192 ± 92* |
| 7 | 2124 ± 293* | 12052 ± 1535 | 1176 ± 96* |
| 8 | 2664 ± 370* | 14534 ± 1540 | 1409 ± 92* |
| 9 | 3152 ± 515* | 16182 ± 1581 | 1373 ± 113 |

### Effects of Starting Treatment with Test Compound Prior to the Development of Renal Complications

URINARY N-ACETYL-β-D-GLUCOSAMINIDASE ACTIVITY
(mU/hour)

| Treatment | Zucker fatty rats | Zucker fatty rats | Lean rats |
|---|---|---|---|
| Duration | Test Compound | Control | Control |
| (months) | (50 μmol/kg of diet) | (powdered chow) | (powdered chow) |
| 0 | 5.7 ± 0.4 | 5.9 ± 0.3 | 4.4 ± 0.3* |
| 1 | 7.5 ± 0.7 | 9.1 ± 0.8 | 5.1 ± 0.5* |
| 2 | 3.8 ± 1.1* | 8.2 ± 1.5 | 6.5 ± 0.3 |
| 3 | 4.5 ± 0.9* | 8.2 ± 1.0 | 5.2 ± 0.4* |
| 4 | 5.1 ± 1.2* | 9.3 ± 0.5 | 6.9 ± 0.5* |
| 5 | 6.5 ± 0.7* | 10.7 ± 0.4 | 7.6 ± 0.7* |
| 6 | 6.5 ± 1.1* | 10.8 ± 1.7 | 7.4 ± 0.5 |
| 7 | 8.9 ± 0.8* | 11.7 ± 0.7 | 7.1 ± 0.5* |
| 8 | 6.2 ± 1.2* | 11.8 ± 1.3 | 7.4 ± 0.5* |
| 9 | 9.1 ± 1.2* | 13.4 ± 0.8 | 7.7 ± 0.8* |

### Effects of Starting Treatment with Test Compound Once Renal Complications have Become Established

SYSTOLIC BLOOD PRESSURE (mm Hg)

| Treatment | Zucker fatty rats | Zucker fatty rats | Lean rats |
|---|---|---|---|
| Duration | Test Compound | Control | Control |
| (months) | (50 μmol/kg of diet) | (powdered chow) | (powdered chow) |
| −4 | 106 ± 3 | 106 ± 3 | 123 ± 3* |
| −3 | 123 ± 5 | 120 ± 4 | 128 ± 3 |
| −2 | 138 ± 7 | 137 ± 4 | 132 ± 4 |
| −1 | 141 ± 6 | 142 ± 6 | 134 ± 3 |
| 0 | 146 ± 4 | 146 ± 4 | 132 ± 4* |
| 1 | 140 ± 5* | 157 ± 6 | 136 ± 4* |
| 2 | 134 ± 3* | 150 ± 6 | 133 ± 2* |
| 3 | 144 ± 3* | 157 ± 6 | 128 ± 6* |
| 4 | 139 ± 3* | 155 ± 4 | 130 ± 3* |
| 5 | 146 ± 6* | 164 ± 4 | 136 ± 3* |

### Effects of Starting Treatment with Test Compound Once Renal Complications have Become Established

URINARY PROTEIN CONCENTRATION (μg/hour)

| Treatment | Zucker fatty rats | Zucker fatty rats | Lean rats |
|---|---|---|---|
| Duration | Test Compound | Control | Control |
| (months) | (50 μmol/kg of diet) | (powdered chow) | (powdered chow) |
| −4 | 220 ± 13 | 287 ± 17 | 198 ± 19* |
| −3 | 347 ± 32 | 355 ± 18 | 321 ± 23* |
| −2 | 457 ± 21 | 509 ± 28 | 396 ± 21* |
| −1 | 958 ± 194 | 874 ± 102 | 393 ± 25* |
| 0 | 3520 ± 905 | 3965 ± 731 | 613 ± 52* |
| 1 | 3692 ± 386* | 5823 ± 809 | 1395 ± 88* |
| 2 | 5326 ± 967* | 8946 ± 1171 | 1192 ± 92* |
| 3 | 6230 ± 835* | 12052 ± 1535 | 1178 ± 96* |
| 4 | 5379 ± 708* | 14534 ± 1540 | 1409 ± 92* |
| 5 | 7677 ± 825* | 16182 ± 1581 | 1373 ± 113* |

### Effects of Starting Treatment with Test Compound Once Renal Complications have Become Established

URINARY N-ACETYL-β-D-GLUCOSAMINIDASE ACTIVITY
(mU/hour)

| Treatment | Zucker fatty rats | Zucker fatty rats | Lean rats |
|---|---|---|---|
| Duration | Test Compound | Control | Control |
| (months) | (50 μmol/kg of diet) | (powdered chow) | (powdered chow) |
| −4 | 5.4 ± 0.2 | 5.9 ± 0.3 | 4.4 ± 0.3* |
| −3 | 8.3 ± 0.5 | 9.1 ± 0.8 | 5.1 ± 0.5* |
| −2 | 9.5 ± 1.1 | 8.2 ± 1.5 | 6.5 ± 0.3 |
| −1 | 7.8 ± 0.6 | 8.2 ± 1.0 | 5.2 ± 0.4* |
| 0 | 7.6 ± 0.7* | 9.3 ± 0.5 | 6.9 ± 0.5* |
| 1 | 7.6 ± 0.7* | 10.7 ± 0.4 | 7.6 ± 0.7* |
| 2 | 5.7 ± 0.9* | 10.8 ± 1.7 | 7.4 ± 0.5 |
| 3 | 7.4 ± 0.9* | 11.7 ± 0.7 | 7.1 ± 0.5* |
| 4 | 5.4 ± 1.0* | 11.8 ± 1.3 | 7.4 ± 0.5* |
| 5 | 8.0 ± 1.1* | 13.4 ± 0.8 | 7.7 ± 0.6* |

1. A method for the treatment and/or prophylaxis of renal diseases including diabetic nephropathy, glomerulonephritis, glomerular sclerosis, nephrotic syndrome, hypertensive nephrosclerosis and end stage renal disease and microalbuminuria which method comprises the administration of an effective, non-toxic amount of an insulin sensitiser to a human or non-human mammal in need thereof.

2. A method according to claim 1, wherein the insulin sensitiser is a thiazolidinedione insulin sensitiser.

3. A method according to claim 1, wherein the insulin sensitiser is an acyclic insulin sensitiser.

4. A method according to claim 1, wherein the insulin sensitiser is a compound of formula (I): 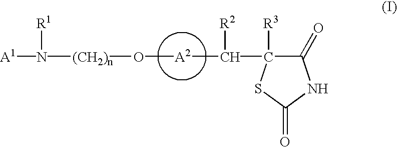
or a tauromeric form thereof and/or a pharmaceutically acceptable salt thereof, and/or a pharmaceutically acceptable solvate thereof, wherein:
A¹ represents a substituted or unsubstituted aromatic heterocyclyl group;
R¹ represents a hydrogen atom, an alkyl group, an acyl group, an aralkyl group,
wherein the aryl moiety may be substituted or unsubstituted, or a substituted or unsubstitured aryl group;
R² and R³ each represent hydrogen, or R² and R³ together represent a bond;
A² represents a benzene ring having in total up to five substituents; and
n represents an integer in the range of from 2 to 6; to a human or non-human mammal in need thereof.

5. A method according to claim 4, wherein in the compound of formula (I) A¹ represents a moiety of formula (a), (b) or (c): 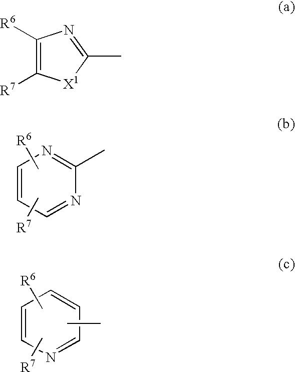
wherein:
R⁶ and R⁷ each independently represents a hydrogen or halogen atom, an alkyl or alkoxy group or a substituted or unsubstituted aryl group or when R⁶ and R⁷ are each attached to adjacent carbon atoms, then R⁶ and R⁷ together with the carbon atoms to which they are attached form a benzene ring wherein each carbon atom represented by R⁶ and R⁷ together is substituted or unsubstituted; and in the moiety of formula (a) X¹ represents oxygen or sulphur.

6. A method according to claim 5, wherein in the compound of formula (I) A¹ represents a moiety of the above defined formula (c). 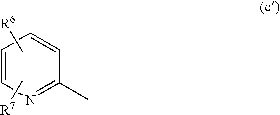
wherein R⁶ and R⁷ are as defined in claim 5.

7. A method according to claim 1, wherein the insulin sensitiser is 5-[4-[2-(N-methyl-N-(2-pyridyl)amino)ethoxy]benzyl]thiazolidine-2,4-dione or a tautomeric form thereof and/or a pharmaceutically acceptable salt thereof and/or a pharmaceutically acceptable solvate thereof.

8. A method according to claim 7, wherein the insulin sensitiser is a maleic acid salt of 5-[4-[2-(N-methyl-N-(2-pyridyl)amino)ethoxy]benzyl]thiazolidine-2,4-dione or a tauromeric form thereof and/or a pharmaceutically acceptable solvate thereof.

9. The use of an insulin sensitiser for the manufacture of a medicament for the treatment and/or prophylaxis of renal diseases including diabetic nephropathy, glomerulonephritis, glornerular sclerosis, nephrotic syndrome, hypertensive nephrosclerosis and end stage renal disease and microalbuminuria.

10. A pharmaceutical composition for the treatment and/or prophylaxis of renal diseases including diabetic nephropathy, glomerulonephritis, glomerular sclerosis, nephrotic syndrome, hypertensive nephrosclerosis and end stage renal disease and microalbuminuria which composition comprises an insulin sensitiser and a pharmaceutically acceptable carrier therefor.
